(19) United States
(12) Patent Application Publication
(10) Pub. No.: US 2025/0127645 A1
(43) Pub. Date: Apr. 24, 2025

(54) BENT KNEE SPLINT

(71) Applicant: Terry L. Whipple, Richmond, VA (US)

(72) Inventor: Terry L. Whipple, Richmond, VA (US)

(21) Appl. No.: 18/922,242

(22) Filed: Oct. 21, 2024

Related U.S. Application Data
(60) Provisional application No. 63/544,861, filed on Oct. 19, 2023.

(51) Int. Cl. A61F 5/058

(52) U.S. Cl. CPC A61F 5/0585

(57) ABSTRACT

A novel temporary knee-splint is disclosed herein. The invention provides an apparatus to immobilize a knee joint following an acute injury in order to maintain the stability of the knee. The knee-splint allows for a variable angle of knee flexion in order to increase comfort. The preferred embodiment of the knee-splint is formed from a thermoplastic polymer and is secured when in use via straps having a hook and loop arrangement. Anchor bars, also secured by the same straps, are used to maintain the desired angle of knee flexion when in use.

This application claims priority from U.S. Provisional Patent Application Nos. 63/544,861 filed on Oct. 19, 2023.

### FIELD OF THE INVENTION

The present invention relates to orthotic devices, namely temporary knee splints which offer stability, protection, and support for injured knee joints.

### BACKGROUND OF THE INVENTION

Acute musculoskeletal injuries to a knee joint and/or associated anatomical structures often require stabilization through immobilization in order to minimize further damage and reduce pain prior to evaluation or treatment. While temporary splints currently exist in the field, such devices possess significant limitations.

Current temporary knee splints for use with an acute injury are typically truncated, cylindrical-like forms that hold the knee in full extension. Acute knee injuries cause effusions and hemarthroses that accumulate in the popliteal sac posteriorly. Straightening the knee joint for current splints is often painful for the patient due to effusion, stretching collateral ligaments or cruciate ligaments, crushing torn menisci, and shifting peri-articular fracture fragments. Immobilization of such an injury in a flexed position is generally more comfortable for many patients, if doing so can also provide the required stability.

There is, therefore, a need for an apparatus which provides the required stability while securing an injured knee in one or more flexed position(s). Such devices should have low manufacturing costs and, further, be easily transported and stored.

### SUMMARY OF THE INVENTION

The present invention teaches a temporary knee-splint or brace for use with acute injuries which allows a knee to be secured at various positions along the knee joint's range of motion. In a preferred embodiment, the knee-splint is formed from a flexible polypropylene sheet having top and bottom portions connected by an articulation point or line. In use, the top and bottom portions of the splint are folded around the affected limb and the entire splint is locked into place through the use of Velcro straps and anchor bars.

The knee splint is able to secure a knee at various angles of knee flexion. This feature allows for immobilization in a desired flexed position which is often more comfortable for many patients having acute knee injuries. The knee-splint disclosed herein is also reasonably inexpensive to fabricate, convenient to store, and may be easily and quickly applied in the field.

### BRIEF DESCRIPTION OF THE DRAWINGS

FIG. 1 is a view of the bottom surface of the invention.

FIG. 2 is a view of the top surface of the invention.

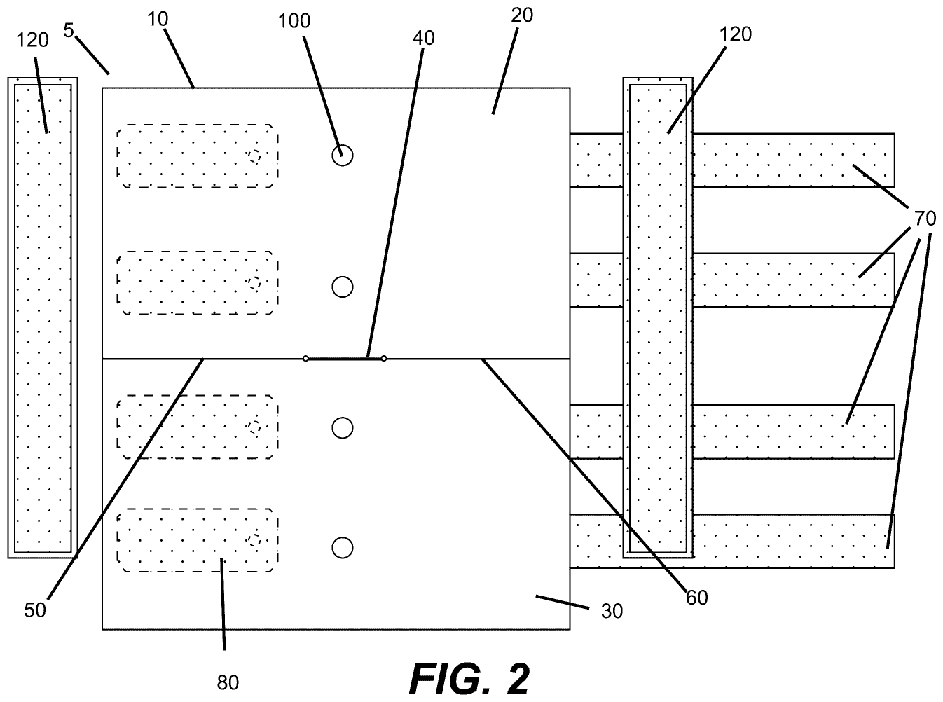

FIG. 3 is a perspective view of the invention when assembled for use.

FIG. 4 is a perspective view of the invention when in use.

FIG. 5 is a perspective rear view of the invention when assembled for use.

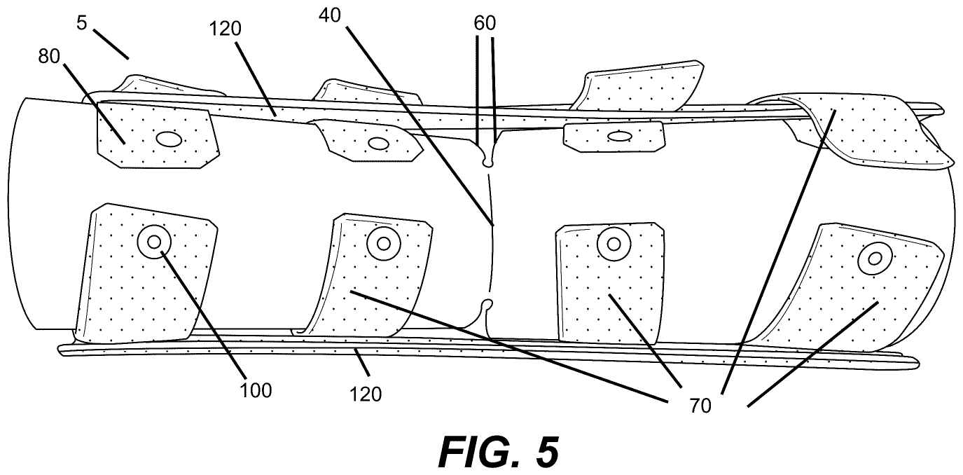

### DETAILED DESCRIPTION

The invention provides a bent knee-splint 5 consisting of a planar polypropylene sheet 10 having an upper portion 20 and a lower portion 30. The upper and lower portions 20, 30, sometimes referred to as proximal section 20 and distal section 30 when in use, are joined along a mid-line joint 40. The two portions 20, 30 are not connected other than at the mid-line 40. Specifically, the edges 50, 60 are juxtaposed when the sheet 10 is lying flat but are not actually connected to one another.

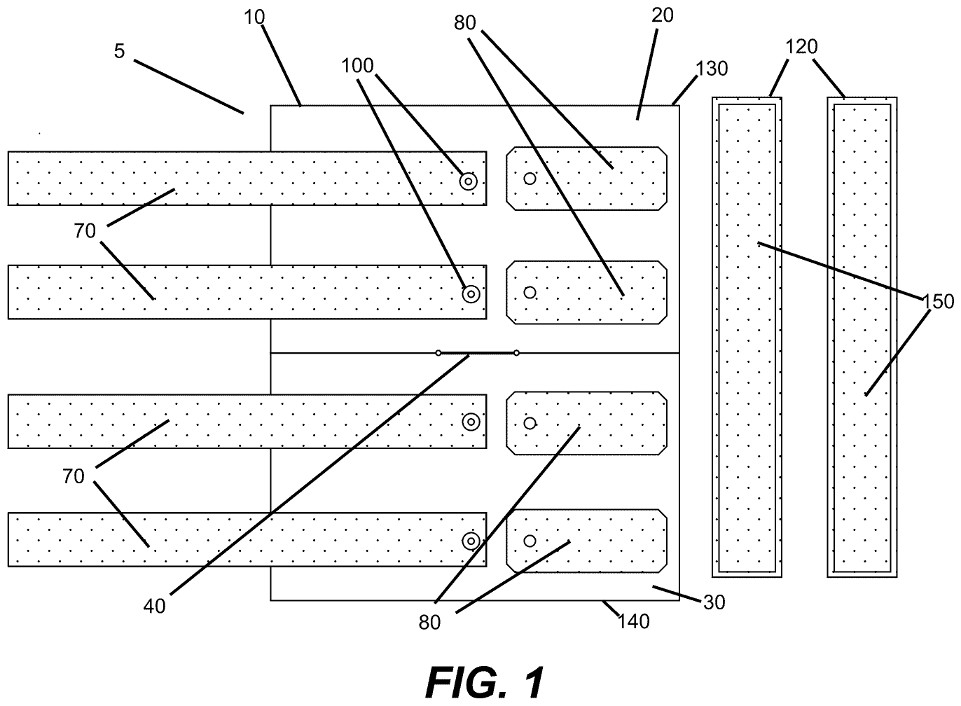

As shown in FIG. 1, the splint includes an attachment mechanism for securing the splint to a patient's limb. This attachment mechanism comprises circumferential belts 70 and a set of attachment pads 80 secured to the rear side 90 of the sheet 10. In the preferred embodiment, two straps 70 and two attachment pads 80 are affixed to each portion of the sheet 10. Further, in such embodiment, the straps 70 are attached to the sheet 10 by a swivel grommet 100 which provides secure fixation while allowing the straps 70 to pivot around the fixation point 100. Any suitable means of attachment may be used as long as such means are functionally equivalent to the grommet 100. Each strap 70 and its corresponding attachment pad 80, i.e. the attachment pad 80 lying parallel to the strap on the sheet 10, comprise a hook and loop fastener arrangement, commonly known as Velcro. The attachment pads 80 may be secured by appropriate fastener or by a sufficiently strong adhesive. No straps or attachment pads lie on the front side 110 of the sheet 10. It will be noted that a greater or lesser number of straps and pads may be used depending on the size of the straps, the size of the splint, and the adhesive strength/resistance to force required.

The splint 5 further includes one or more side anchors 120. Each side anchor 120 is a flat, rectangular shaped bar which is approximately the length of the long-axis, i.e., from the top of the sheet 130 to the bottom of the sheet 140, of the sheet 10. The side anchors 120 are fabricated from polypropylene or the same material as the sheet 10. The preferred embodiment would include two side anchors 120. The majority of each face of a side anchor 120 has an attachment portion 150 (consisting of Velcro hook or loop portions, as appropriate) affixed thereto. In some cases, the attachment portion 150 would cover the side anchor 120 almost in its entirety, as seen in FIG. 1. However, it will be recognized that the attachment portion 150 may cover less than the majority of the anchor-again, as dictated by the required adhesive strength.

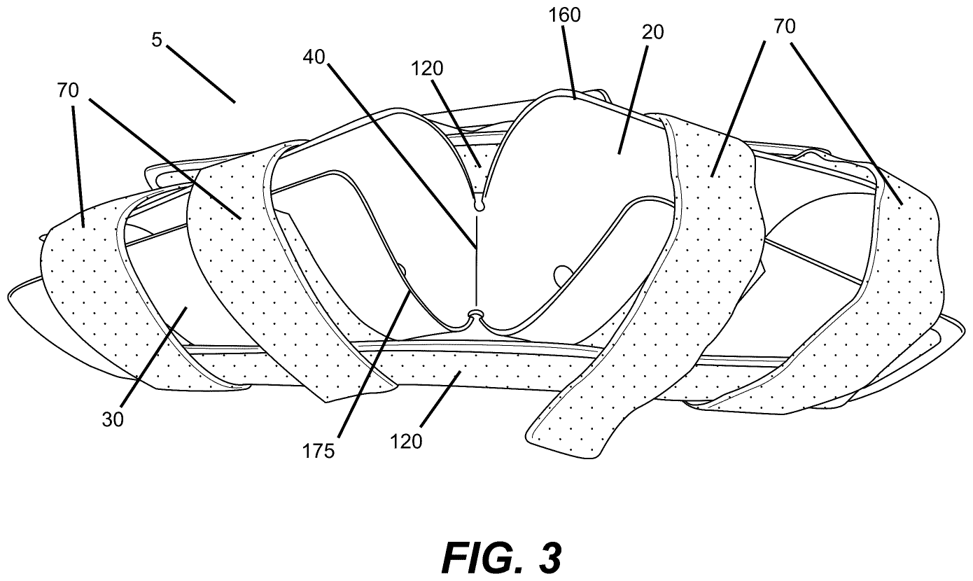

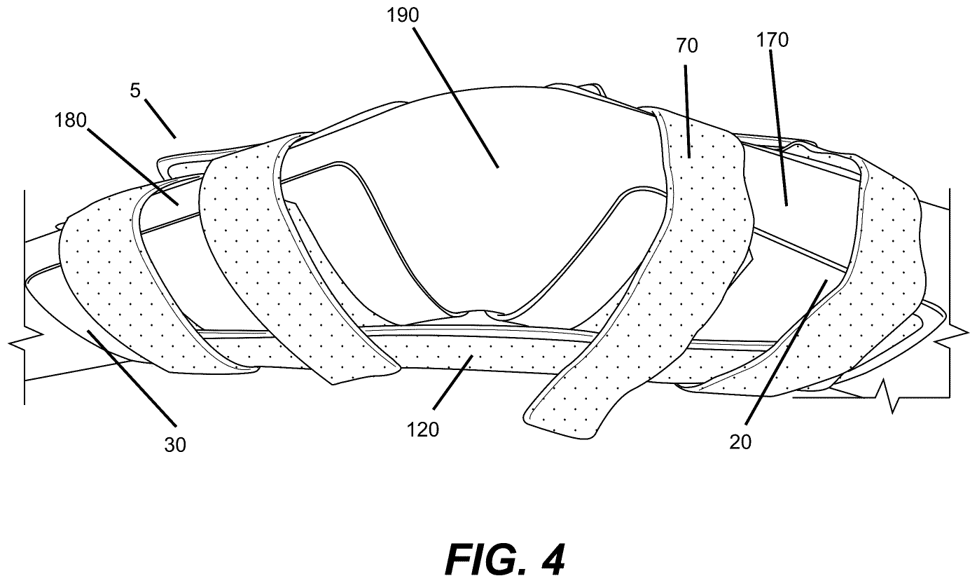

Turning to FIGS. 3 and 4, the splint is used to immobilize the knee joint of the wearer. In use, the upper portion 20 is bent or folded into a proximal U-shaped form 160 to encompass the upper leg 170 (above the knee) of the wearer. The lower portion 30 is bent or folded into a distal U-shaped form 175 to encompass the lower leg 180 (below the knee) of the wearer. The mid-line joint 40 serves as an articulation point or hinge for the splint 5 and, accordingly, the knee 190 of the wearer.

Once the desired angle of knee flexion is set, the side anchors 120 are secured in place on either side of the splint 5. Specifically, when attached, the circumferential belts 70 and the anchors maintain the U-shape around the leg and secure the splint to the leg of the wearer. Moreover, the belts and anchors lock the brace in place and prevent further movement at the articulation point or any change in the angle of knee flexion. The attachment arrangement also prevents travel up or down the leg while being worn. By adjusting knee flexion, the leg may be immobilized within a range of angles, from an essentially extended position to a flexed position approaching ninety degrees.

The invention herein provides numerous advantages and benefits over prior art braces. The splint may be applied swiftly without moving the knee of the patient appreciably during application. When in use, does not apply pressure on collateral ligaments or knee condyles thereby increasing comfort and decreasing any adverse effects. The use of flexible sheets allows the brace to more easily accommodate legs' of varying size or girth. Flat, open splints 5 may be easily stored in stacks reducing storage space on shelves, in ambulances, or in any storage location. Finally, fabrication costs are generally lower than more advanced braces or braces composed of other materials. Indeed, fabrication costs would be considerably less than those of braces composed of metal or with extensive metal components.

In the preferred embodiment, a polypropylene sheet is used. However, the sheet 10 may also be formed from any thermoplastic polymer which possess the desired characteristics. The sheet must be of such material and thickness to have sufficient flexibility and strength so that it can be bent or molded into the required U-shaped form around the patient's leg while in use but still provide adequate stability for the knee joint. The length of the sheets may vary but in the preferred embodiment would be approximately eighteen to twenty inches in maximum length, top to bottom, and fourteen to sixteen inches in maximum width, side-to-side.

An alternate embodiment of the invention provides a knee splint of similar design comprised of paperboard. This embodiment is formed from a somewhat trapezoid shape paperboard having parallel horizontal cuts on either side of a pre-creased fold line at the approximate mid-level of the splint. The pre-creased fold line serves as the articulation point for knee flexion and, as above, divides the paperboard splint into a top portion and a bottom portion. Additionally, a set of two pre-creased fold lines run along the long axis of the splint within both the upper and lower portions allowing the sides to fold-up in order to form a U-shape so as to encircle three sides of the wearer's leg when in use. Circumferential belts would be used to secure the splint to an extremity. Typically, the belts would consist of two belts around a thigh and two or three belts around calf of a user.

The belts in this embodiment are secured to the midline extending along the length of the splint with swivel capability and attach with Velcro pads, or any equivalent means, on ends of the belts. In use, this embodiment of the splint is locked in place through the use of a stiff, rectangular side anchor also comprised of paperboard of analogous weight which attaches with a Velcro surface to the bent splint over the opened, midlevel cuts on splint sides, securing them in a variable angle of knee flexion. It will be noted that any analogous locking mechanism may be used to secure the two sections of the splint when in use. The paperboard knee splint is largely biodegradable which is ideal for a one-use type product. Further, a splint of this material may also be fabricated quickly and rather inexpensively.

While the invention has been described in reference to certain preferred embodiments, it will be readily apparent to one of ordinary skill in the art that certain modifications or variations may be made to the device without departing from the scope of invention described in the foregoing specification.

1. An adjustable knee splint for stabilizing a knee joint comprising:
an upper U-shaped proximal cuff formed from a thermoplastic polymer sheet having an interior side and an exterior side and further having at least one attachment strap and at least one attachment pad affixed thereto; said upper cuff being circumscribed by said at least one attachment strap;
a lower U-shaped distal cuff formed from a thermoplastic polymer sheet having an interior side and an exterior side and further having at least one attachment strap and at least one attachment pad affixed thereto; said lower cuff being circumscribed by said at least one attachment strap;
said upper cuff and said lower cuff partially connected by an articulated portion positioned at a pre-determined angle; and,
at least one side anchor positioned along the long axis of said knee splint on said inner side and at least one side anchor positioned on said outer side; said side anchors being removably secured in place by said at least one attachment strap or said at least one attachment pad on said respective upper cuff and lower cuff, whereby said articulation portion is locked at a fixed angle thereby preventing travel of the knee joint outside the locked angle.

2. The knee splint of claim 1 where said thermoplastic polymer is polypropylene.

3. The knee splint of claim 1 wherein said at least one attachment strap comprises two or more attachment straps and, further, wherein said at least one attachment pad comprises two or more attachment pads.

4. The knee splint of claim 3 wherein said two or more attachment straps are attached to said respective upper cuff and lower cuff by at least one fastener for each respective strap.

5. The knee splint of claim 4 wherein said fastener is a swivel grommet.

6. An adjustable knee splint for stabilizing a knee joint comprising:
a planar thermoplastic polymer sheet having an upper section and a lower section separated by an edge; at least a portion of said upper and said lower sections being unconnected and at least a portion of said upper and said lower sections being connected at said edge by a hinge member;
each of said upper and lower sections having at least one attachment strap and at least one attachment pad secured thereto; said at least one attachment strap and said at least one attachment pad having a hook and loop fastener arrangement thereon such that said strap may engage said pad;
said upper section and said lower section being formed into independent U-shaped structures when in use which are secured in place by said at least one attachment strap and at least one attachment pad on the respective upper section and lower sections; and,
an outside anchor support removably affixed on a first side of the splint to at least a portion of both said upper and said lower sections and an inside anchor support removably affixed on a second side of the splint to at least a portion of both said upper and said lower sections,
whereby said hinge member of the brace is locked at a fixed angle to prevent unwanted flexion of the knee joint.

7. The adjustable knee splint of claim 6 wherein more than one said splint may be stacked essentially flat upon one another for storage purposes when not in use.

8. The adjustable knee splint of claim 6 wherein said thermoplastic polymer sheet is comprised of polypropylene.

9. An adjustable knee splint for stabilizing a knee joint comprising:
a rigid paperboard sheet having an upper portion and a lower portion defined by a pre-creased articulation line; said sheet further possessing two additional pre-creased fold lines which extend parallel to the edges of the sheet along the long axis of the sheet from said upper portion to said lower portion on either side of a center-line of the sheet, thereby permitting the sheet to be folded into two independent u-shaped sections comprising a first u-shaped section and a second u-shaped section;
at least one belt, affixed to said upper portion, circumscribing said first u-shaped section and secured via hook and loop fastener means;
at least one belt, affixed to said lower portion, circumscribing said second u-shaped section and secured via hook and loop fastener means; and,
at least one side anchor removably affixed to both said upper portion and said lower portion such that it prevents movement of the articulation line.
